(19) United States
(12) Patent Application Publication
(10) Pub. No.: US 2011/0152936 A1
(43) Pub. Date: Jun. 23, 2011

(54) DIRECTIONAL VERTEBRAL ROD

(75) Inventors: Carlos E. Gil, Collierville, TN (US); Aleksandr G. Zolotov, Collierville, TN (US)

(73) Assignee: Warsaw Orthopedic, Inc., Warsaw, IN (US)

(21) Appl. No.: 12/643,273

(22) Filed: Dec. 21, 2009

(51) Int. Cl. A61B 17/70

(52) U.S. Cl. 606/259

(57) ABSTRACT

A vertebral rod includes a first elongated section having a first thinned portion. A second elongated section has a second thinned portion. An intermediate section has a flat, thin configuration, and is connected with and disposed between the first section and the second section. The flat, thin configuration of the intermediate section is disposed in an orientation transverse to at least one of the first thinned portion and the second thinned portion.

### TECHNICAL FIELD

The present disclosure generally relates to medical devices for the treatment of spinal disorders, and more particularly to a dynamic vertebral rod system, having multiple directional capability, which provides stability while reducing stress on spinal elements.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders include discectomy, laminectomy, fusion and implantable prosthetics. As part of these surgical treatments, connecting elements such as vertebral rods are often used to provide stability to a treated region. During surgical treatment, one or more rods may be attached to the exterior of two or more vertebral members.

Rods redirect stresses away from a damaged or defective region while healing takes place to restore proper alignment and generally support the vertebral members. In some applications, rods are attached to the vertebral members without the use of implants or spinal fusion. Flexible connecting elements are also known that permit limited spinal motion of a spinal motion segment. Such flexible connecting elements can provide dynamic spinal support.

The present disclosure describes improvements over these prior art technologies.

### SUMMARY

Accordingly, a dynamic vertebral rod system is provided, having single or multiple directional capability. It is contemplated that such capability can include movement in flexion, extension, lateral bending and rotation to provide stability while reducing stress on spinal elements.

In one embodiment, a vertebral rod of the present disclosure includes a first elongated section having a first thinned portion and a second elongated section having a second thinned portion. An intermediate section, having a flat, thin configuration, is connected with and disposed between the first section and the second section. The flat, thin configuration of the intermediate section is disposed in an orientation transverse to at least one of the first thinned portion and the second thinned portion.

In one embodiment, the vertebral rod includes a first elongated section, a second elongated section and a discoid intermediate section. The discoid intermediate section has a continuous outer surface and is connected with and disposed between the first and second sections. At least a portion of the intermediate section is flexible.

In one embodiment, the vertebral rod has a first elongated section defining a first axis and a second elongated section defining a second axis. A discoid intermediate section is connected with and disposed between the first section and the second sections. The discoid intermediate section is flexible and has an elliptical configuration that defines an elongated axis.

### BRIEF DESCRIPTION OF THE DRAWINGS

The present disclosure will become more readily apparent from the specific description accompanied by the following drawings, in which:

FIG. 1 is a perspective view of one particular embodiment of a vertebral rod in accordance with the principles of the present disclosure;

FIG. 2 is a plan view of the vertebral rod shown in FIG. 1;

FIG. 3 is a side view of the vertebral rod shown in FIG. 1;

FIG. 4 is a perspective view of a vertebral rod system including the vertebral rod shown in FIG. 1 attached to vertebrae;

FIG. 5 is a lateral section view of the vertebral rod system attached to vertebrae;

FIG. 6 is a perspective view of one embodiment of the vertebral rod of the present disclosure;

FIG. 7 is a side view of the vertebral rod shown in FIG. 6;

FIG. 8 is a plan view of one embodiment of the vertebral rod of the present disclosure;

FIG. 9 is a side view of the vertebral rod shown in FIG. 8; and

FIG. 10 is a perspective view of one embodiment of the vertebral rod of the present disclosure;

Like reference numerals indicate similar parts throughout the figures.

### DETAILED DESCRIPTION

The exemplary embodiments of the vertebral rod system and methods of use disclosed are discussed in terms of medical devices for the treatment of spinal disorders and more particularly, in terms of a dynamic vertebral rod having multiple directional capability. Such capability can include flexion, extension, lateral bending and rotational movement. It is envisioned that the vertebral rod system and methods of use disclosed provide stability and maintains structural integrity while reducing stress on spinal elements. It is contemplated that a vertebral rod of the system can maintain structural integrity in an axial direction of the vertebral rod while reducing stress in a radial direction of the vertebral rod.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is further envisioned that the present disclosure may be employed with surgical treatments including open surgery and minimally invasive procedures of such disorders, such as, for example, discectomy, laminectomy, fusion, bone graft and implantable prosthetics. It is contemplated that the present disclosure may be employed with other osteal and bone related applications, including those associated with diagnostics and therapeutics. It is further contemplated that the disclosed vertebral rod system may be employed in a surgical treatment with a patient in a prone or supine position, employing a posterior, lateral or anterior approach. The present disclosure may be employed with procedures for treating the lumbar, cervical, thoracic and pelvic regions of a spinal column. The system and methods of the present disclosure may also be used on animals, bone models and other non-living substrates, such as for training, testing and demonstration.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right are for illustrative purposes only and can be varied within the scope of the present disclosure. For example, the references “upper” and “lower” are relative and used only in the context to the other, and are not necessarily “superior” and “inferior”.

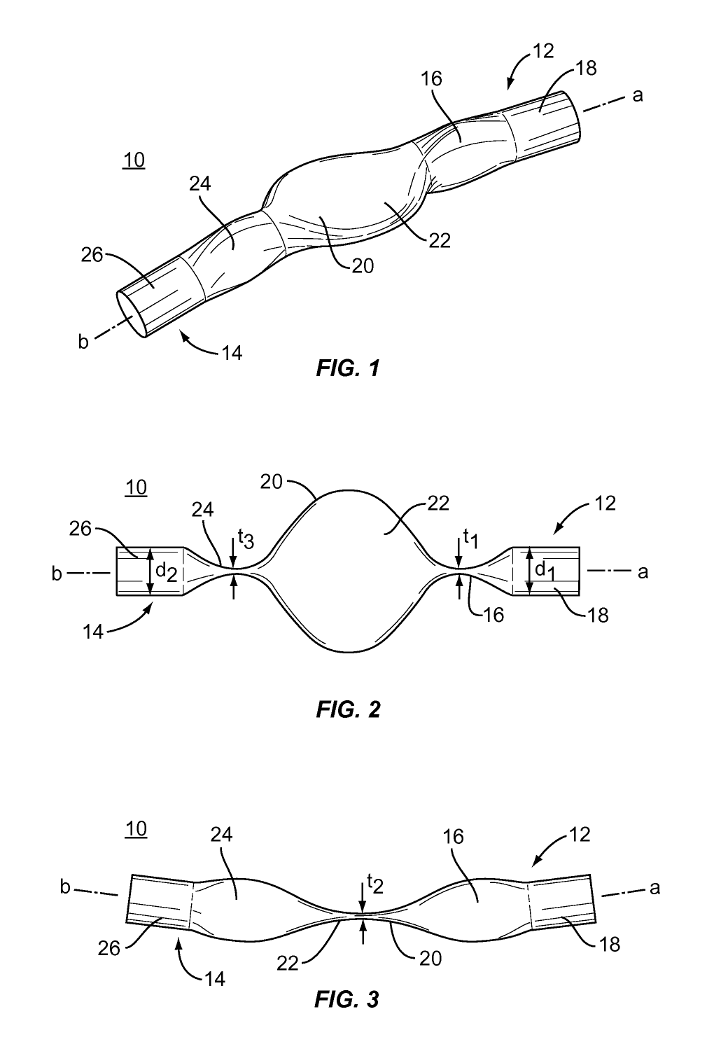

The following discussion includes a description of a vertebral rod system, related components and exemplary methods of employing the vertebral rod system in accordance with the principles of the present disclosure. Alternate embodiments are also disclosed. Reference will now be made in detail to the exemplary embodiments of the present disclosure, which are illustrated in the accompanying figures. Turning now to FIGS. 1-5, there are illustrated components of a vertebral rod system in accordance with the principles of the present disclosure.

The components of the vertebral rod system are fabricated from materials suitable for medical applications, including metals, polymers, ceramics, biocompatible materials and/or their composites, depending on the particular application and/or preference of a medical practitioner. For example, a vertebral rod, discussed below, of the vertebral rod system can be fabricated from materials such as commercially pure titanium, titanium alloys, super-elastic titanium alloys, cobalt-chrome alloys, cobalt-chrome-molybdenum alloys, stainless steel alloys, super elastic metallic alloys (e.g., Nitinol, super elasto-plastic metals, such as GUM METAL® manufactured by Toyotsu Material Incorporated of Japan), shape memory materials, thermoplastics such as polyaryletherketone (PAEK) including polyetheretherketone (PEEK), polyetherketoneketone (PEKK) and polyetherketone (PEK), continuous carbon fiber reinforced PEEK and/or short carbon fiber reinforced PEEK composites, PEEK-BaSO₄ composites, biocompatible materials such as polymers including plastics, metals, ceramics and composites thereof, rigid polymers including polyphenylene, polyamide, polyimide, polyetherimide, polyethylene, polyurethane, epoxy, silicone; and different sections of the rod may have alternative material composites to achieve various desired characteristics such as strength, rigidity, elasticity, compliance, biomechanical performance, durability and radiolucency or imaging preference. It is contemplated that the vertebral rod may employ a heterogeneous composite of the materials described and may have a non-uniform carbon content.

It is envisioned that the vertebral rod can be manufactured via various methods including machining, casting, injection-molding, insert-molding, overmolding, compression molding, transfer molding, co-extrusion, pultrusion, dip-coating, spray-coating, powder-coating, porous-coating and their combinations. One skilled in the art, however, will realize that such materials and fabrication methods suitable for assembly and manufacture, in accordance with the present disclosure, would be appropriate.

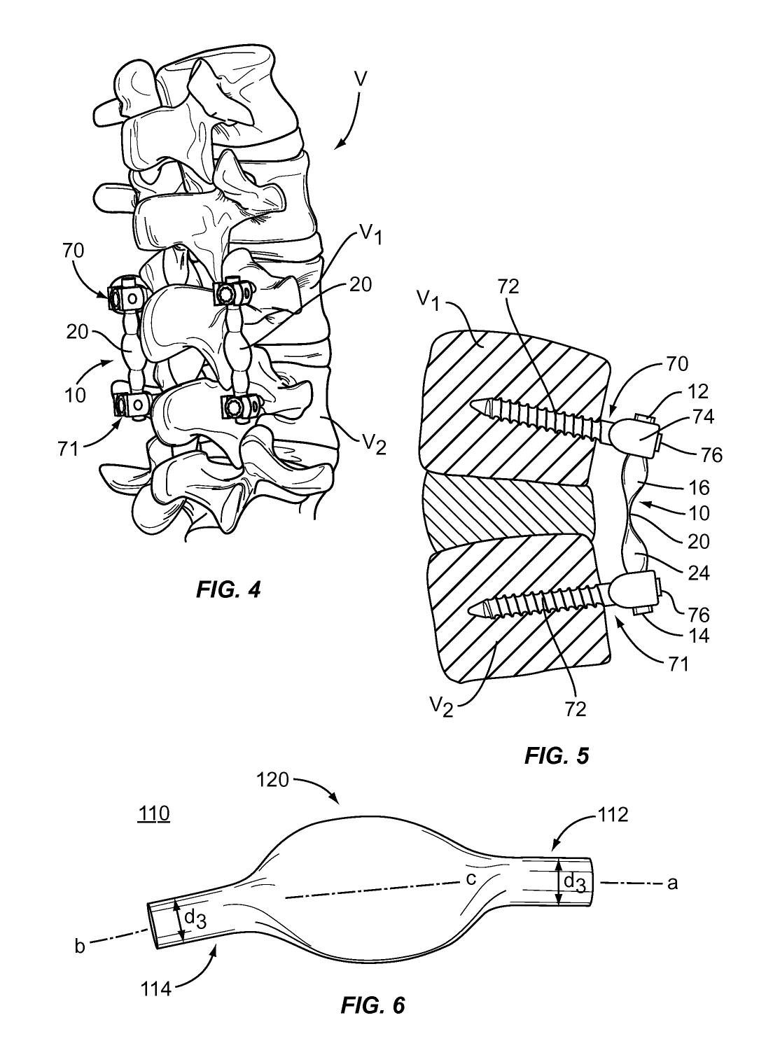

The vertebral rod system is configured for attachment to vertebrae (as shown, for example, in FIGS. 4 and 5) during surgical treatment of a spinal disorder, examples of which are discussed herein. The vertebral rod system has a vertebral rod 10, which includes a first elongated section, such as, for example, upper section 12 that defines a longitudinal axis a. A second elongated section, such as, for example, lower section 14 defines a longitudinal axis b.

Upper section 12 includes a first thinned portion 16 that is flexible and an end portion 18. End portion 18 has a uniform diameter d₁ and first thinned portion 16 defines a first thickness, such as, for example, a minimum thickness t₁. First thinned portion 16 gradually decreases from diameter d₁ to minimum thickness t₁ such that first thinned portion 16 has a flattened configuration and gradually increases therefrom to the intermediate section.

The flattened configuration of first thinned portion 16 facilitates lateral bending of upper section 12 in either lateral direction about the area adjacent minimum thickness t₁ along an axis defined thereby, which is traverse to axis a. The flattened configuration of first thinned portion 16 may also facilitate rotational twisting of upper section 12. The area adjacent minimum thickness t₁ may have a planar outer surface. The cross section of rod 10 adjacent thickness t₁ may be uniform, non-uniform, spherical, staggered and/or slotted. It is envisioned that all or only a portion of upper section 12 is flexible. It is contemplated that diameter d₁ may be in a range of approximately 4-8 millimeters (mm). In one embodiment, vertebral rod 10 includes a titanium alloy and diameter d₁ is approximately 4.75 mm. It is further contemplated that minimum thickness t₁ may be in a range of approximately 0.3-2.0 mm. In one embodiment, vertebral rod 10 includes a titanium alloy and thickness t₁ is approximately 0.6 mm. Depending on the material(s) employed, a minimal thickness is desirable to resist axial loads while maintaining stability to prevent buckling of vertebral rod 10.

First thinned portion 16 has a reduced thickness and increased width that provides greater flexibility to upper section 12 and rod 10 in a radial direction while simultaneously maintaining structural integrity in support of an axial load to upper section 12 and rod 10. This configuration facilitates movement of a spine, while preventing undesirable compression of vertebral bodies, for example, preventing loading of articular facet joints.

A discoid intermediate section 20 is connected with sections 12, 14 and disposed therebetween as a joining section of the components of vertebral rod 10. Intermediate section 20 has a thin, flat disk configuration and is connected to first portion 16. Intermediate section 20 is circular and defines a minimum thickness t₂ corresponding to the thin, flat disk configuration. The thin, flat disc configuration of intermediate section 20 is flexible and bendable in flexion and extension about the area adjacent minimum thickness t₂, along an axis defined thereby, which is transverse to axes a, b. The discoid configuration of intermediate section 20 may also facilitate rotational twisting of vertebral rod 10. It is contemplated that a minimal thickness t₂ resists axial loads while maintaining stability to prevent buckling of vertebral rod 10.

Thickness t₂ is disposed in a transverse orientation relative to thickness t₁. Thickness t₂ may be disposed in an orientation relative to thickness t₁ including perpendicular, at an acute angular orientation, such as, for example, 75 degrees, and/or parallel. It is contemplated that minimum thickness t₂ of intermediate section 20 may be in a range of approximately 0.4-2.4 mm and in one embodiment thickness t₂ is approximately 0.5 mm. Intermediate section 20 may have a variable thickness t₂ according to the requirements of the particular application. The cross section of rod 10 adjacent thickness t₂ may also be uniform, non-uniform, spherical, staggered and/or slotted.

Intermediate section 20 has a reduced thickness and increased width that provides greater flexibility to intermediate section 20 and rod 10 in a radial direction while simultaneously maintaining structural integrity of an axial load to intermediate section 20 and rod 10. This configuration facilitates movement of a spine while preventing undesirable compression of vertebral bodies.

Intermediate section 20 defines a substantially planar outer surface 22. Outer surface 22 may alternatively include all or portions thereof having texture, undulations and/or dimpled portions. Outer surface 22 has opposing planar sides. Alternatively, one of the sides may be planar and the other non-planar. It is envisioned that outer surface 22 may have machined surfaces, polished surfaces, smooth surfaces, textured surfaces, shot-peened surfaces, burnished surfaces, porous surfaces, patterned surfaces and wavy surfaces. Outer surface 22 may be chemically treated or modified using various processes or materials that include oxidation, anodization, plasma treatment, vapor deposition, plating, coating and etching.

Lower section 14 includes a second thinned portion 24 that is flexible and an end portion 26. End portion 26 has a uniform diameter d₂ and second thinned portion 24 defines a minimum thickness t₃. Second thinned portion 24 gradually decreases from intermediate section 20 to minimum thickness t₃ such that second thinned portion 24 has a flattened configuration and gradually increases therefrom to diameter d₂. Diameter d₂ is equal to diameter d₁, however, diameter d₂ and d₁ may be nonequal and/or offset.

The flattened configuration of second thinned portion 24 facilitates lateral bending of lower section 14 in either lateral direction about the area adjacent minimum thickness t₃ along an axis defined thereby, which is transverse to axis b. Thickness t₃ is disposed in a transverse orientation relative to thickness t₂ and in a parallel orientation relative to thickness t₁. Thickness t₃ may be disposed in an orientation relative to thickness t₁ and/or t₂ including perpendicular, at an acute angular orientation, such as, for example, 75 degrees, and/or parallel.

The flattened configuration of second thinned portion 24 may also facilitate rotation/twisting of lower section 14. The area adjacent minimum, thickness t₃ may have a planar outer surface. The cross section of rod 10, adjacent thickness t₃ may be uniform, non-uniform, spherical, staggered and/or slotted. It is envisioned that all or only a portion of lower section 14 is flexible. It is contemplated that diameter d₂ may be in a range of approximately 4-8 mm. In one embodiment, vertebral rod 10 includes a titanium alloy and diameter d₂ is approximately 4.75 mm. It is further contemplated that minimum thickness t₃ may be in a range of approximately 0.3-2.0 mm. In one embodiment, vertebral rod 10 includes a titanium alloy and thickness t₃ is approximately 0.6 mm. Similar to first thinned portion 16, the configuration of second thinned portion 24 facilitates movement of a spine while preventing undesirable compression of vertebral bodies.

It is envisioned that the components of vertebral rod 10 may be monolithically formed, integrally connected or arranged with attaching elements. Intermediate section 20 is flexible and configured to provide resistance to movement of sections 12, 14. Intermediate section 20 may provide increasing, variable, constant and/or decreasing resistance. It is contemplated that sections 12, 14, 20 can be variously dimensioned, for example, with regard to length, width, diameter and thickness. It is further contemplated that the respective cross-section of sections 12, 14, 20 may have various configurations, for example, round, oval, rectangular, irregular, uniform and non-uniform. Section 12 may have a different cross-sectional area, geometry, material or material property such as strength, modulus or flexibility relative to section 14. It is envisioned that the cross-sectional geometry or area of intermediate section 20 can be uniform, non-uniform, consistent or variable.

Intermediate section 20 may have one or a plurality of elements connecting sections 12, 14 such as spaced apart portions, staggered patterns and mesh. Intermediate section 20 may be fabricated from the same or alternative material to sections 12, 14. Intermediate section 20 may also have a different cross-sectional area, geometry or material property such as strength, modulus and flexibility relative to sections 12, 14. Intermediate section 20 may be connected to sections 12, 14 using various methods and structure including molding of a continuous component, mechanical fastening, adhesive bonding and combinations thereof.

It is envisioned that particular parameters may be selected to modulate the flexibility or stiffness of the vertebral rod system including the cross-sectional area (or thickness) of intermediate section 20. These parameters allow modification of the properties or performance of the vertebral rod system such as strength, durability, flexibility (or stiffness), overall profile and the ability to employ a percutaneous approach, for a particular application. In one embodiment, sections 12, 14, 20 are fabricated from a stainless steel alloy, such as, for example, BioDur® 108 Alloy manufactured by Carpenter Technology Corporation. In one embodiment, sections 12, 14, 20 are fabricated from a titanium alloy, such as, for example, CP Titanium or Ti-6Al-4V.

It is contemplated that vertebral rod 10 may include one or a plurality of intermediate sections 20 spaced along the length of rod 10. In embodiments including a plurality of sections 20, the multiple sections 20 may be disposed in similar, or alternative orientations such as aligned, non-aligned, offset, open end facing or not facing vertebrae and alternate angular orientation. It is envisioned that only one of sections 12, 14 have a thinned portion, as described, or alternatively, that section 12 and/or section 14 may include one or a plurality of thinned portions.

In assembly, operation and use, the vertebral rod system is employed with a surgical procedure for treatment of a spinal disorder affecting a section of a spine of a patient, as discussed herein. The vertebral rod system may also be employed with other surgical procedures. In particular, the vertebral rod system is employed with a surgical procedure for treatment of a condition or injury of an affected section of the spine including vertebrae V, as shown in FIGS. 4 and 5. It is contemplated that the vertebral rod system is attached to vertebrae V for dynamic stabilization of the affected section of the spine to provide stability for healing and therapeutic treatment, while allowing a desirable range of motion or load-sharing capability.

In use, to treat the affected section of the spine, a medical practitioner obtains access to a surgical site including vertebra V in any appropriate manner, such as through incision and retraction of tissues. It is envisioned that the vertebral rod system may be used in any existing surgical method or technique including open surgery, mini-open surgery, minimally invasive surgery and percutaneous surgical implantation, whereby the vertebrae V is accessed through a mini-incision, or sleeve that provides a protected passageway to the area. Once access to the surgical site is obtained, the particular surgical procedure is performed for treating the spinal disorder. The vertebral rod system is then employed to augment the surgical treatment. The vertebral rod system can be delivered or implanted as a pre-assembled device or can be assembled in situ. The vertebral rod system may be completely or partially revised, removed or replaced.

A first fastening element, such as, for example, fixation screw assembly 70 is configured to attach upper section 12 to vertebra V₁. A second fastening element, such as, for example, fixation screw assembly 71 is configured to attach lower section 14 to adjacent vertebra V₂. Pilot holes are made in vertebrae V₁, V₂ for receiving fixation screw assemblies 70, 71. Fixation screw assemblies 70, 71 include threaded bone engaging portions 72 that are inserted or otherwise connected to vertebrae V₁, V₂, according to the particular requirements of the surgical treatment. Fixation screw assemblies 70, 71 each have a head 74 with a bore, or through opening and a set screw 76, which is torqued on to sections 12, 14 to attach rod 10 in place with vertebrae V, as will be described.

As shown in FIGS. 4 and 5, the vertebral rod system includes two axially aligned and spaced rods 10, with portions of sections 12, 14 extending through the bores of heads 74. Set screws 76 of each head 74 are torqued on the end portions of rods 10 to securely attach rods 10 with vertebrae V₁, V₂. Upon fixation of the vertebral rod system with vertebrae V, vertebral rod 10 is configured to provide increasing resistance to multi-directional movement of sections 12, 14 during flexion, extension, lateral bending and/or rotation of the spine. For example, when in an unloaded or neutral state, there is no appreciable tensile or compressive loads on the spinal motion segment comprising vertebrae V₁, V₂ and the intervertebral disc in between, or on vertebral rod 10. During movement of the spinal motion segment caused by corresponding movement of the patient, rod 10 reacts with increasing resistance during movement of rod 10 to a plurality of orientation(s) due, for example, to flexion, extension, lateral bending and/or rotation/twisting of vertebrae V.

For example, in flexion, extension, lateral bending and/or rotation, upper section 12 moves relative to section 14. In flexion and extension, intermediate section 20 bends or collapses about an axis transverse to axes a, b such that outer surface 22 folds in a direction facing vertebrae V (flexion) or in a direction opposing vertebrae V (extension). In lateral bending, first thinned portion 16 and/or second thinned portion 24 bend or collapse about axes transverse to axes a, b, respectively, such that sections 12, 14 fold in either lateral direction depending on patient movement. In rotation, intermediate section 20 and/or first thinned portion 16 and/or second thinned portion 24 rotate or twist about axes a, b depending on patient movement. For example, if vertebrae V is caused to rotate clockwise on its own axis, intermediate section 20 and/or first thinned portion 16 and/or second thinned portion 24 rotate clockwise about axes a, b. If vertebrae V is caused to rotate counter clockwise, intermediate section 20 and/or first thinned portion 16 and/or second thinned portion 24 rotate counter-clockwise about axes a, b.

This configuration of intermediate section 20, first thinned portion 16 and second thinned portion 24 increases resistance during multi-directional movement, which includes flexion, extension, lateral bending and/or rotation. The increase of resistance during flexion, extension, lateral bending and/or rotation provides limited movement of vertebrae V for dynamic stabilization of the treated area of the spine. The configuration of rod 10 also provides support of vertebral bodies in any axial direction. It is contemplated that dynamic stabilization can be provided for various patient movement, which can be compensated with various combinations of reaction from rod 10 including bending of intermediate section 20, bending of first thinned portion 16 and/or second thinned portion 24, rotation/twisting of intermediate section 20, rotation/twisting of first thinned portion 16 and/or rotation/twisting of second thinned portion 24. It is envisioned that variation of the thickness of the cross section of rod 10 adjusts stiffness of rod 10 for treatment of a particular pathology and/or patent application.

The vertebral rod system can be used with various bone screws, pedicle screws or multi-axial screws used in spinal surgery. It is contemplated that the vertebral rod system may be used with pedicle screws coated with an osteoconductive material such as hydroxyapatite and/or osteoinductive agent such as a bone morphogenic protein for enhanced bony fixation to facilitate motion of the treated spinal area. Rod 10 can be made of radiolucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used, such as being disposed at end portions 18, 26 of rod 30 and/or along the length thereof adjacent intermediate section 20.

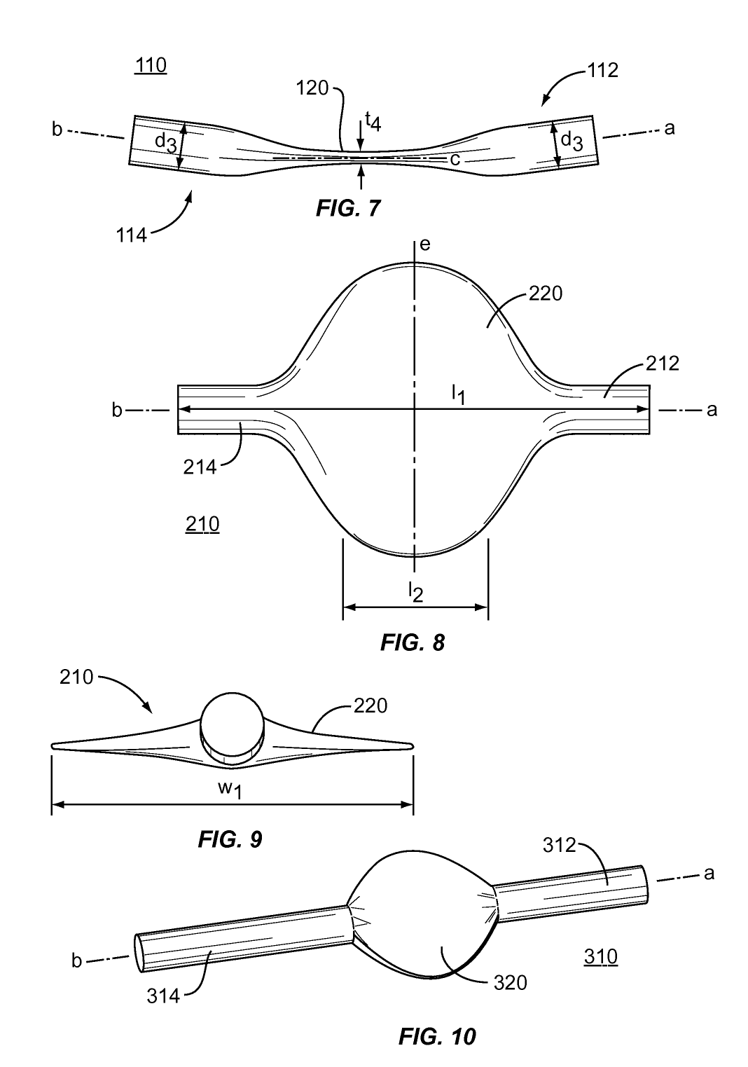

Referring to FIGS. 6 and 7, in one embodiment similar to vertebral rod 10 described above, a vertebral rod 110 includes an upper section 112 that defines a longitudinal axis a and a lower section 114 that defines a longitudinal axis b. Sections 112, 114 define a uniform diameter d₃. A discoid intermediate section 120 is connected with and disposed between sections 112, 114. Intermediate section 120 is flexible and has an elliptical configuration that defines an elongated axis c. Axis c is oriented substantially co-axial with axes a, b. It is contemplated that axis c may be offset, traverse or angularly disposed relative to axis a and/or axis b. Intermediate section 120, similar to section 20 described above, defines a thickness t₄ that may be in a range of approximately 0.4-2.5 mm and in one embodiment thickness t₄ is approximately 0.5 mm.

Referring to FIGS. 8 and 9, in one embodiment similar to vertebral rod 110 described above, a vertebral rod 210 has a discoid intermediate section 220 that is connected with and disposed between sections 212, 214. Section 220 is flexible and has an elliptical configuration that defines an elongated axis e. Axis e is orientated substantially traverse to axes a, b. Vertebral rod 210 defines a length I₁ which in one embodiment is approximately 44.6 mm. Intermediate section 220 defines a length I₂, which corresponds to the flat portion and/or minimum thickness of section 220. Length I₂ can be varied to affect the stiffness of rod 210 for treatment of a particular pathology and/or patient application. It is envisioned that I₂ may be in a range of approximately 1-10 mm.

Vertebral rod 210 also defines a width w₁ that may be in a range of approximately 10-30 mm and in one embodiment width w₁, is approximately 28 mm.

Referring to FIG. 10, in one embodiment similar to vertebral rod 10 described above, a vertebral rod 310 includes an upper section 312 that defines a longitudinal axis a and a lower section 314 that defines a longitudinal axis b. Sections 312, 314 define uniform diameters respectively. A discoid intermediate section 320 is connected with and disposed between sections 312, 314. Section 320 is flexible and has a circular configuration.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A vertebral rod comprising:
a first elongated section having a first thinned portion,
a second elongated section having a second thinned portion; and
an intermediate section having a flat, thin configuration, and being connected with and disposed between the first section and the second section,
the flat, thin configuration of the intermediate section being disposed in an orientation transverse to at least one of the first thinned portion and the second thinned portion.

2. A vertebral rod according to claim 1, wherein the first thinned portion and the second thinned portion are each disposed in a substantially perpendicular orientation relative to the flat, thin configuration of the intermediate section.

3. A vertebral rod according to claim 1, wherein the first thinned portion and the second thinned portion are each directly connected to the intermediate section.

4. A vertebral rod according to claim 1, wherein at least a portion of the intermediate section is flexible.

5. A vertebral rod according to claim 1, wherein the first elongated section has an end portion connected to the first thinned portion and the second elongated section has an end portion connected to the second thinned portion, the end portions having uniform diameters.

6. A vertebral rod according to claim 1, wherein the intermediate section defines a discoid including the flat, thin configuration.

7. A vertebral rod according to claim 6, wherein the discoid intermediate section has a circular configuration.

8. A vertebral rod according to claim 6, wherein the discoid intermediate section has an elliptical configuration.

9. A vertebral rod according to claim 1, wherein the sections are fabricated from a titanium alloy.

10. A vertebral rod according to claim 1, wherein the sections are fabricated from a stainless steel alloy.

11. A vertebral rod comprising:
a first elongated section;
a second elongated section; and
a discoid intermediate section having a continuous outer surface and being connected with and disposed between the first and second sections, at least a portion of the intermediate section being flexible.

12. A vertebral rod according to claim 11, the first elongated section including a first thinned portion and the second elongated section including a second thinned portion, the discoid intermediate section being disposed in an orientation transverse to at least one of the first thinned portion and the second thinned portion.

13. A vertebral rod according to claim 11, wherein the discoid intermediate section has a circular configuration.

14. A vertebral rod according to claim 11, wherein the discoid intermediate section has an elliptical configuration.

15. A vertebral rod according to claim 11, wherein the sections are fabricated from a titanium alloy.

16. A vertebral rod according to claim 11, wherein the sections are fabricated from a stainless steel alloy.

17. A vertebral rod comprising:
a first elongated section defining a first axis;
a second elongated section defining a second axis; and
a discoid intermediate section connected with and disposed between the first section and the second section, the discoid intermediate section being flexible and having an elliptical configuration that defines an elongated axis.

18. A vertebral rod according to claim 17, wherein the elongated axis of the elliptical configuration is substantially coaxial with the first axis and the second axis.

19. A vertebral rod according to claim 17, wherein the elongated axis of the elliptical configuration is orientated substantially transverse to the first axis and the second axis.

20. A vertebral rod according to claim 17, wherein the first elongated section includes a first thinned portion and the second elongated section includes a second thinned portion, the discoid intermediate section being disposed in an orientation transverse to at least one of the first thinned portion and the second thinned portion.
